(19) United States
(12) Patent Application Publication
(10) Pub. No.: US 2026/0209147 A1
(43) Pub. Date: Jul. 23, 2026

(54) OXIDATIVE DEHYDROGENATION WITH WASTEWATER-DERIVED OXYGEN

(71) Applicant: NOVA CHEMICALS (INTERNATIONAL) S.A., Fribourg (CH)

(72) Inventors: Vasily SIMANZHENKOV, Calgary (CA); Ce YANG, Clarksville, MD (US); Shahin GOODARZNIA, Calgary (CA); Sina MIRZAEIFARD, Calgary (CA)

(21) Appl. No.: 19/138,210

(22) PCT Filed: Nov. 14, 2023
(86) PCT No.: PCT/IB2023/061510

§ 371 Date: Jun. 12, 2025

Related U.S. Application Data
(60) Provisional application No. 63/432,536, filed on Dec. 14, 2022.

(51) Int. Cl. C07C 5/48; B01J 6/00; B01J 8/04; C01B 3/38; C01B 3/56; C25B 1/04; H01M 8/0656

(52) U.S. Cl. CPC C07C 5/48; B01J 6/008; B01J 8/0492; C01B 3/38; C01B 3/56; H01M 8/0656; C01B 2203/0233; C01B 2203/042; C01B 2203/048; C01B 2203/065; C01B 2203/066; C25B 1/04

(57) ABSTRACT

The present disclosure relates to oxidative dehydrogenation of ethane-containing feed streams with oxygen derived from electrolysis of wastewater.

### TECHNICAL FIELD

The present disclosure relates to oxidative dehydrogenation of ethane-containing feed streams with oxygen derived from electrolysis of wastewater.

### BACKGROUND ART

Catalytic oxidative dehydrogenation of alkanes into corresponding alkenes is an alternative to steam cracking. In contrast to steam cracking, oxidative dehydrogenation (ODH) can operate at lower temperatures and generally does not produce coke. For ethylene production, oxidative dehydrogenation can provide a greater selectivity for ethylene than steam cracking. However, oxidative dehydrogenation requires oxygen, which is typically provided by energy-intensive air separation. Additionally, certain processes for preparing an oxidative dehydrogenation feedstock, such as hydrocracking, require hydrogen, which is typically provided by steam methane reforming, which produces carbon dioxide, a greenhouse gas. Integration of other low-carbon sources of hydrogen, such as hydrogen generated by renewable energy (green hydrogen) or by reduced-emissions processes (blue hydrogen) can be economically infeasible, for example, due to increased capital costs, increased operating costs, and/or decreased reliability. Accordingly, there is a need for improved, integrated low-carbon oxidative dehydrogenation processes.

### SUMMARY OF INVENTION

Provided in the present disclosure is a method of producing ethylene, the method including contacting an oxidative dehydrogenation feed stream including ethane and an oxidant stream including oxygen with an oxidative dehydrogenation catalyst to form a dehydrogenated stream including ethylene and water; separating the dehydrogenated stream to form a product stream including ethylene and a waste stream including water; and electrolyzing at least a portion of the waste stream including water to form a first electrolysis stream including oxygen and a second electrolysis stream including hydrogen. The oxidant stream includes at least a portion of the first electrolysis stream including oxygen.

In some embodiments, the method further includes, before contacting the oxidant stream with the oxidative dehydrogenation catalyst, separating oxygen from air to form an air-separated stream including oxygen, and the oxidant stream includes at least a portion of the air-separated stream including oxygen.

In some embodiments, the method further includes oxidizing at least a portion of a fuel cell feed stream including at least a portion of the second electrolysis stream including hydrogen to produce electrical energy, and electrolyzing at least a portion of the waste stream uses at least a portion of the electrical energy from oxidizing the fuel cell feed stream.

In some embodiments, the method further includes heating a steam cracking feed stream including a hydrocarbon and water to form a cracked stream including ethane (e.g., unreacted ethane) and a fuel gas including hydrogen, and the oxidative dehydrogenation feed stream includes a portion of the cracked stream including ethane. In some embodiments, the method further includes combusting at least a portion of the second electrolysis stream including hydrogen to produce thermal energy, and using at least a portion of the thermal energy to heat the steam cracking feed stream. In some embodiments, the method further includes oxidizing at least a portion of a fuel cell feed stream including a portion of the cracked stream including the fuel gas to produce electrical energy, and electrolyzing at least a portion of the waste stream uses at least a portion of the electrical energy from oxidizing the fuel cell feed stream.

In some embodiments, the fuel gas further includes methane. In some embodiments, the method further includes separating at least a portion of the methane from the fuel gas. In some embodiments, the method further includes contacting a hydrocracking feed stream including a hydrocarbon and hydrogen with a hydrocracking catalyst to form a hydrocracked stream including ethane, and the steam cracking feed stream includes at least a portion of the hydrocracked stream including ethane. In some embodiments, the hydrocracking feed stream includes at least a portion of the second electrolysis stream including hydrogen.

In some embodiments, the hydrocracking feed stream includes at least a portion of the cracked stream including the fuel gas.

In some embodiments, the hydrocracking feed stream includes a pyrolysis oil. In some embodiments, the pyrolysis oil includes a treated pyrolysis oil. In some embodiments, the method further includes contacting a hydrotreating feed stream including a raw pyrolysis oil and a portion of the second electrolysis stream including hydrogen with a hydrotreating catalyst to form the treated pyrolysis oil. In some embodiments, the method further includes pyrolyzing a plastic to form the raw pyrolysis oil. In some embodiments, the pyrolyzing includes thermal pyrolysis, hydrothermal pyrolysis, or catalytic pyrolysis.

In some embodiments, oxidizing the fuel cell feed stream includes contacting the fuel cell feed stream with an anode catalyst of a fuel cell. In some embodiments, the method further includes storing a portion of the first electrolysis stream including hydrogen, storing at least a portion of the second electrolysis stream including oxygen, or both.

In some embodiments, the method further includes combusting an oxy-fuel combustion mixture including a portion of the first electrolysis stream including oxygen, and a fuel feed stream including a combustible fuel to produce thermal energy and carbon dioxide; converting at least a portion of the thermal energy to electrical energy; and capturing at least a portion of the carbon dioxide. In some embodiments, the method further includes contacting a gas stream including a pollutant with a portion of the first electrolysis stream including oxygen to oxidize at least a portion of the pollutant.

In some embodiments, electrolyzing at least a portion of the waste stream uses electrical energy from a renewable energy source or a reduced-emissions process.

Also provided in the present disclosure is a system for producing ethylene, the system including an oxidative dehydrogenation reactor configured to contact an oxidative dehydrogenation feed stream including ethane and an oxidant stream including oxygen with an oxidative dehydrogenation catalyst to form a dehydrogenated stream including ethylene and water; a separator configured to separate the dehydrogenated stream to form a product stream including ethylene and a waste stream including water; and an electrolyzer configured to electrolyze at least a portion of the waste stream including water to form a first electrolysis stream including oxygen and a second electrolysis stream including hydrogen, and to provide at least a portion of the first electrolysis stream to the oxidative dehydrogenation reactor.

In some embodiments, the system further includes an air separation unit configured separate oxygen from air to form an air-separated stream including oxygen, and to provide at least a portion of the air-separated stream to the oxidative dehydrogenation reactor.

In some embodiments, the system further includes a fuel cell configured to oxidize at least a portion of a fuel cell feed stream to produce electrical energy, and to provide at least a portion of the electrical energy to the electrolyzer.

In some embodiments, the system further includes a steam cracking reactor configured to heat a steam cracking feed stream including a hydrocarbon and water to form a cracked stream including ethane and a fuel gas including hydrogen, and to provide a portion of the cracked stream including the fuel gas to the oxidative dehydrogenation reactor. In some embodiments, the system further includes a pressure swing adsorption unit configured to separate methane from the fuel gas.

In some embodiments, the system further includes a hydrocracking reactor configured to contact a hydrocarbon and hydrogen with a hydrocracking catalyst to form a hydrocracked stream including ethane, and to provide a portion of the hydrocracked stream including ethane to the steam cracking reactor.

In some embodiments, the system further includes a hydrotreating reactor configured to contact a hydrotreating feed stream including a raw pyrolysis oil and a portion of the second electrolysis stream including hydrogen with a hydrotreating catalyst to form a treated pyrolysis oil, and to provide a portion of the treated pyrolysis oil to the hydrocracking reactor. In some embodiments, the system further includes a reactor configured to pyrolyze a plastic to form the raw pyrolysis oil.

### BRIEF DESCRIPTION OF DRAWINGS

FIG. 1 is a schematic diagram of a system for producing ethylene.

FIG. 2 is a process flow diagram of a method for producing ethylene.

FIG. 3 is a set of schematic illustrations of (top) a process according to certain embodiments described herein and (bottom) a comparative process described herein.

FIG. 4 is a set of schematic illustrations of (top) a process according to certain embodiments described herein and (bottom) a comparative process described herein.

FIG. 5 is a set of schematic illustrations of (top) a process according to certain embodiments described herein and (bottom) a comparative process described herein.

FIG. 6 is a set of schematic illustrations of (top) a process according to certain embodiments described herein and (bottom) a comparative process described herein.

### DESCRIPTION OF EMBODIMENTS

The present disclosure relates to methods and systems for ethylene production by oxidative dehydrogenation integrated with water electrolysis. The operational methods and systems described herein provide a process for producing ethylene by oxidative dehydrogenation of ethane using oxygen formed by electrolyzing wastewater from the oxidative dehydrogenation process, including co-produced water and other process water (e.g., water used to scrub the oxidative dehydrogenation reactor effluent). In certain embodiments, the methods and systems produce less carbon dioxide as compared to those including integrated steam-methane reforming and/or air separation processes. In certain embodiments, the methods and systems require less energy as compared to those including integrated steam-methane reforming and/or air separation processes. The methods and systems described herein can further include integrated low-carbon sources of power, such as a fuel cell and/or oxy-combustion power plant, to drive the integrated water electrolysis.

FIG. 1 is a schematic illustration of a system 100 for producing ethylene in accordance with certain embodiments of the present disclosure. The system 100 includes an oxidative dehydrogenation reactor 102, a separator 104, and an electrolyzer 106.

Liquid and gaseous streams and materials used in the system represented in FIG. 1 can be directed and transferred through suitable transfer lines, conduits, and piping. Particular elements may be physically juxtaposed and, where appropriate, may have flexible regions, rigid regions, or a combination of both. In directing streams of compounds, intervening apparatuses and/or optional treatments may be included. For example, pumps, valves, manifolds, gas and liquid flow meters and distributors, sampling and sensing devices, and other equipment (e.g., for monitoring, controlling, adjusting, and/or diverting pressures, flows and other operating parameters) can be included in the system.

An oxidant stream 101 including oxygen (O₂) and an oxidative dehydrogenation feed stream 103 including ethane (C₂H₆) are directed to the oxidative dehydrogenation reactor 102 including an oxidative dehydrogenation catalyst (not shown). Suitable configurations of the oxidative dehydrogenation reactor are known in the art. For example, the oxidative dehydrogenation reactor can include one shell-and-tube reactor, or two or more shell-and-tube reactors (e.g., in series and/or in parallel). In some embodiments, the oxidant stream, the oxidative dehydrogenation feed stream, or both include a diluent. In some examples, the diluent can help to control the heat of reaction released. Certain diluents can be reactive, and can change the selectivity of ethylene. The diluent can include, for example, steam (H₂O), carbon dioxide (CO₂), nitrogen (N₂), helium (He), argon (Ar), and the like. In some embodiments, the diluent includes steam, carbon dioxide, or both.

The composition of the oxidant stream and the oxidative dehydrogenation feed stream can be selected to provide to the reactor, in combination, a mixture including about 10 wt. % to about 90 wt. % of ethane. The composition of the oxidant stream and the oxidative dehydrogenation feed stream can be selected to provide to the reactor, in combination, a mixture including about 10 wt. % to about 90 wt. % of oxygen. In certain such embodiments, the oxidant stream and the dehydrogenation feed stream are free from diluents.

The composition of the oxidant stream and the oxidative dehydrogenation feed stream can be selected to provide to the reactor, in combination, a mixture including about 1 wt. % to about 40 wt. % oxygen, e.g., about 1 wt. % to about 30 wt. %, about 1 wt. % to about 25 wt. %, about 2 wt. % to about 20 wt. %, or about 5 wt. % to about 15 wt. % oxygen. For example, the composition of the oxidant stream and the oxidative dehydrogenation feed stream can be selected to provide to the reactor, in combination, a mixture including about 1 wt. % to about 40 wt. % of ethane for example, about 1 wt. % to about 35 wt. %, about 2.5 wt. % to about 30 wt. %, or about 5 wt. % to about 25 wt. % of ethane. In certain such embodiments, the mixture further includes a diluent including steam, carbon dioxide, or both.

The oxidant stream and/or the oxidative dehydrogenation stream can include trace amounts (e.g., less than 1 wt. %) of typical pipeline ethane contaminants such as propane, C₄-C₁₀ hydrocarbons, methanol, H₂S, and the like. Where water is used as a diluent, typical water feed contaminants, such as acetic acid, ethanol, and heavier oxygenates, can also be present.

In some embodiments, the oxidative dehydrogenation feed stream includes the product of a hydrocracking process. For example, in some embodiments, the oxidative dehydrogenation feed stream includes the product of hydrocracking a pyrolysis oil, e.g., derived from catalytic pyrolysis of a plastic. In some embodiments, the oxidative dehydrogenation feed stream includes unreacted ethane from a steam cracking process, and/or ethane from an external source, such as a natural gas processing plant. Other suitable sources of ethane-containing feed streams are known in the art.

Non-limiting examples of the oxidative dehydrogenation catalyst include those containing one or more mixed metal oxides. In some embodiments, the catalyst is selected from:
i) catalysts of the formula:

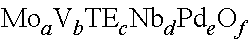

where a, b, c, d, e and f are the relative atomic amounts of the elements Mo, V, Te, Nb, Pd and O, respectively; and where a=1, b=0.01 to 1.0, c=0.01 to 1.0, d=0.01 to 1.0, 0.00≤e≤0.10 and f is a number to at least satisfy the valence state of the metals in the catalyst;
ii) catalysts of the formula:

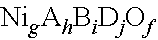

where g is a number from 0.1 to 0.9, such as from 0.3 to 0.9, from 0.5 to 0.85, or from 0.6 to 0.8; h is a number from 0.04 to 0.9; i is a number from 0 to 0.5; j is a number from 0 to 0.5; and f is a number to at least satisfy the valence state of the catalyst; A is chosen from Ti, Ta, V, Nb, Hf, W, Y, Zn, Zr, Si and Al or mixtures thereof; B is chosen from La, Ce, Pr, Nd, Sm, Sb, Sn, Bi, Pb, TI, In, Te, Cr, Mn, Mo, Fe, Co, Cu, Ru, Rh, Pd, Pt, Ag, Cd, Os, Ir, Au, Hg, and mixtures thereof; D is chosen from Ca, K, Mg, Li, Na, Sr, Ba, Cs, and Rb and mixtures thereof; and O is oxygen;
iii) catalysts of the formula:

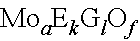

where E is chosen from Ba, Ca, Cr, Mn, Nb, Ta, Ti, Te, V, W and mixtures thereof; G is chosen from Bi, Ce, Co, Cu, Fe, K, Mg, V, Ni, P, Pb, Sb, Si, Sn, Ti, U, and mixtures thereof; a=1; k is 0 to 2; 1=0 to 2, with the proviso that the total value of 1 for Co, Ni, Fe and mixtures thereof is less than 0.5; and f is a number to at least satisfy the valence state of the metals in the catalyst;
iv) catalysts of the formula:

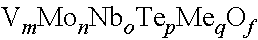

where Me is chosen from Ta, Ti, W, Hf, Zr, Sb and mixtures thereof; m is from 0.1 to 3; n is from 0.5 to 1.5; o is from 0.001 to 3; p is from 0.001 to 5; q is from 0 to 2; and f is a number to at least satisfy the valence state of the metals in the catalyst; and
v) catalysts of the formula:

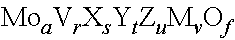

where X is at least one of Nb and Ta; Y is at least one of Sb and Ni; Z is at least one of Te, Ga, Pd, W, Bi and Al; M is at least one of Fe, Co, Cu, Cr, Ti, Ce, Zr, Mn, Pb, Mg, Sn, Pt, Si, La, K, Ag and In; a=1.0 (normalized); r=0.05 to 1.0; s=0.001 to 1.0; t=0.001 to 1.0; u=0.001 to 0.5; v=0.001 to 0.3; and f is a number to at least satisfy the valence state of the metals in the catalyst.
vi) a mixed metal oxide having the empirical formula:

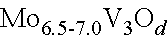

where d is a number to at least satisfy the valence of the metals in the catalyst; and
vii) a mixed metal oxide having the empirical formula:

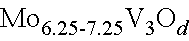

where d is a number to at least satisfy the valence of the metals in the catalyst.

In some embodiments, the catalyst is supported on or agglomerated with a binder. Some binders include acidic, basic, or neutral binder slurries of TiO₂, ZrO₂Al₂O₃, AlO(OH) and mixtures thereof. Another useful binder includes Nb₂O₅. The agglomerated catalyst can be extruded in a suitable shape, such as rings, spheres, or saddles, among others, of a size typically used in fixed bed reactors. When the catalyst is extruded, various extrusion aids known in the art can be used. In some cases, the resulting support may have a cumulative surface area of less than 35 m²/g as measured by BET, in some cases, less than 20 m²/g, in other cases, less than 3 m²/g. and a cumulative pore volume from 0.05 to 0.50 cm³/g.

The oxidative dehydrogenation feed stream and oxidant stream can be contacted with the oxidative dehydrogenation catalyst under any condition suitable for the oxidative dehydrogenation reaction. For example, in some embodiments, the feed stream, oxidant stream, and catalyst are contacted at a temperature of between about 300° C. and about 500° C., or between about 300° C. and about 450° C., or between about 330° C. and about 425° C. In various embodiments, the feed stream, oxidant stream, and catalyst are contacted at a pressure of between about 10 psia to about 175 psia, or between about 25 psia to about 100 psia. In various embodiments, the residence time of the reactor is between about 0.002 seconds and about 30 seconds, or between about 1 seconds and about 10 seconds. In various embodiments, the gas hourly space velocity (GHSV) of the oxidative dehydrogenation reactor is between about 500 h⁻¹ and about 30000 h⁻¹, or greater than about 1000 h⁻¹. Other conditions such as flow rates upstream and downstream of the reactor can be derived from these parameters based on applicable equipment geometries (e.g., reactor volume, catalyst bed size, etc.).

Ethane from the feed stream 103 is dehydrogenated to form a dehydrogenated stream 105 including ethylene (C₂H₄) and water. In some embodiments, the dehydrogenated stream includes about 5 wt. % to about 75 wt. % of ethylene. In some embodiments, the dehydrogenated stream includes about 5 wt. % to about 80 wt. % of water. In some embodiments, the dehydrogenated stream further includes ethane, oxygen, carbon dioxide (CO₂), carbon monoxide (CO), acetic acid, or any combination thereof. For example, in some embodiments, the dehydrogenated stream includes about 0.1 wt. % to about 10 wt. % of carbon monoxide, about 1 wt. % to about 30 wt. % ethane, about 0 wt. % to about 1 wt. % oxygen, and about 0 wt. % to about 10 wt. % acetic acid. Diluent and other trace contaminants (e.g., described herein) and derivatives thereof can also be present in the dehydrogenated stream.

The dehydrogenated stream 105 is separated in separator 104 to form a gaseous product stream 107 including ethylene and a liquid waste stream 109 including water. Separators suitable for gas-liquid separations are known in the art. In some embodiments, the separator includes a condenser (e.g., a shell-and-tube heat exchanger). The separator can include multiple separation units, such as a quench vessel, one or more condensing heat exchangers, and any other extraction or separation equipment suitable to separate co-products from water (e.g., acetic acid). In some embodiments, the separator includes a flash tank. In some embodiments, the separator includes a scrubber, for example, a quench tower, a spray tower, a venture scrubber, or a packed tower. In certain such embodiments, the waste stream includes water fed to the scrubber (e.g., as scrubbing liquid).

The product stream can include at least a portion of gaseous components of the dehydrogenated stream. For example, in some embodiments, the product stream further includes carbon monoxide, carbon dioxide, ethane, or any combination thereof. The waste stream can include at least a portion of condensable components of the dehydrogenated stream. For example, in some embodiments, the waste stream further includes acetic acid. In some embodiments, at least a portion of acetic acid present in the dehydrogenated stream is recovered from the waste stream in the separator, by any suitable separation technology. For example, acetic acid can be separated from the waste stream by distillation or liquid-to-liquid extraction (e.g., before electrolyzing the waste stream water). In certain embodiments, the waste stream includes less than about 0.5 wt. % of acetic acid.

The waste stream 109 is directed to the electrolyzer 106. Water from the waste stream is electrolyzed in electrolyzer 106 to form a first electrolysis stream 111 including oxygen and a second electrolysis stream 113 including hydrogen (H₂). The first electrolysis stream and second electrolysis stream can each independently include one or more trace contaminants (e.g., as described herein) present in the waste stream, or derivatives thereof (e.g., electrolysis products thereof). Suitable electrolyzers are known in the art. For example, in some embodiments, the electrolyzer includes an alkaline electrolyzer, a polymer electrolyte membrane electrolyzer, a solid oxide electrolyzer, or a membrane-less electrolyzer. In some embodiments, the electrolysis is driven by renewable energy or energy from a reduced-emissions process, e.g., solar energy, wind energy, geothermal energy, nuclear energy, waste heat electricity recovery, oxy-fuel combustion with carbon capture, etc.

At least a portion of the first electrolysis stream 111 including oxygen is directed to the oxidative dehydrogenation reactor 102 via oxidant stream 101. In some embodiments, the oxidant stream includes substantially all of the first electrolysis stream. In some embodiments, the oxidant stream further includes oxygen from an air separation unit. In some embodiments, supplementing the oxidant stream with oxygen from an air separation unit increases the capacity of the oxidative dehydrogenation reactor. Supplemental oxygen from an air separation unit can also be used to compensate for diminished output of the electrolyzer (e.g., due to intermittent renewable power). Suitable methods and systems for air separation are known in the art. For example, in some embodiments, the oxidant stream includes oxygen from a cryogenic air separation unit.

In some embodiments, a portion of the first electrolysis stream including oxygen is stored, e.g., as a compressed gas or as a liquid. For example, excess oxygen can be stored when the oxygen demand of the oxidative dehydrogenation reactor is less than the electrolysis output. In such examples, stored oxygen can later be used to supplement the oxidant stream when the oxygen demand of the oxidative dehydrogenation reactor is greater than the electrolysis output (e.g., due to increased availability of oxidative dehydrogenation feedstock, or diminished electrolysis output).

In some embodiments, a portion of the first electrolysis stream including oxygen is directed to an oxy-combustion power plant. For example, in some embodiments, an oxy-fuel combustion mixture including a portion of the first electrolysis stream including oxygen and a fuel feed stream including a combustible fuel is combusted to produce thermal energy and carbon dioxide. At least a portion of the thermal energy can be converted to mechanical and, in some examples, electrical energy, and at least a portion of carbon dioxide formed by combusting the oxy-fuel combustion mixture can be captured. In some embodiments, at least a portion of the electrical energy is used to drive electrolysis of the waste stream water. Oxy-combustion power plants can be configured and operated as known in the art. For example, in some embodiments, the oxy-combustion power plant includes an oxy-fired broiler, an oxy-fired gasifier, or an oxy-fired gas turbine.

In some embodiments, a portion of the first electrolysis stream including oxygen is directed to a thermal oxidizer. For example, in some embodiments, a gas stream including a pollutant (e.g., a volatile organic compound, a hazardous air pollutant, or any combination thereof) is contacted with a portion of the first electrolysis stream to oxidize at least a portion of the pollutant. Thermal oxidizers can be configured and operated as known in the art.

In some embodiments, at least a portion of the second electrolysis stream including hydrogen is directed to a fuel cell. For example, in some embodiments, at least a portion of a fuel cell feed stream including at least a portion of the second electrolysis stream including hydrogen is oxidized to produce electrical energy. In some embodiments, oxidizing the fuel cell feed stream includes contacting the fuel cell feed stream with an anode catalyst of the fuel cell. At least a portion of this electrical energy can be used to drive electrolysis of the waste stream water. Suitable hydrogen fuel cells are known in the art. For example, in some embodiments, the fuel cell includes a polymer electrolyte membrane fuel cell.

In some embodiments, at least a portion of the second electrolysis stream including hydrogen is directed to a hydrocracking reactor. For example, in some embodiments, a hydrocracking feed stream including a hydrocarbon and at least a portion of the second electrolysis stream including hydrogen are contacted with a hydrocracking catalyst to form a hydrocracked stream including ethane. Suitable methods and systems for hydrocracking are known in the art. For example, in some embodiments, the hydrocracking reactor includes an adiabatic multiple-bed reactor (e.g., a fixed-bed or trickle-bed reactor) with interstage cooling. Other hydrocracking reactors include, for example, heat exchanger-type reactors. In some embodiments, the hydrocracking feed stream includes a treated pyrolysis oil.

In some embodiments, at least a portion of the second electrolysis stream including hydrogen is directed to a steam cracking reactor. For example, in some embodiments, at least a portion of the second electrolysis stream including hydrogen is combusted to produce thermal energy. The thermal energy can be used to heat a steam cracking feed stream including a hydrocarbon and water to form a cracked stream including a fuel gas including hydrogen (e.g., and optionally further including one or more C₁-C₂ hydrocarbons). In some embodiments, the fuel gas includes at least about 50 wt. % hydrogen, for example about 50 wt. % to about 100 wt. %, about 60 wt. % to about 100 wt. %, or about 60 wt. % to about 85 wt. % of hydrogen. In some embodiments, the fuel gas further includes methane, for example, up to about 50 wt. % methane, about 5 wt. % to about 40 wt. %, or about 10 wt. % to about 35 wt. % methane. Suitable methods and systems for steam cracking are known in the art. In some embodiments, the steam cracking feed stream includes at least a portion of a hydrocracked stream described herein.

In some embodiments, a portion of the cracked stream including the fuel gas is directed to a fuel cell or a hydrocracking reactor. The fuel gas can be separated from the cracked stream using any suitable method. For example, fuel gas including hydrogen and methane can be separated from the cracked stream by cryogenic distillation (e.g., in a demethanizer column downstream from the steam cracking reactor). In certain such embodiments, the hydrogen content of the fuel gas can be further increased, for example, by pressure swing adsorption (e.g., before introduction to the fuel cell or hydrocracking reactor).

In some embodiments, a portion of a fuel cell feed stream including the fuel gas is oxidized to produce electrical energy. In some embodiments, oxidizing the fuel cell feed stream includes contacting the fuel cell feed stream with an anode catalyst of the fuel cell. At least a portion of this electrical energy can be used to drive electrolysis of the waste stream water. Suitable hydrogen fuel cells are known in the art. For example, in some embodiments, the fuel cell includes a polymer electrolyte membrane fuel cell.

In some embodiments, a portion of the cracked stream including the fuel gas is directed to a hydrocracking reactor (e.g., as described herein). For example, in some embodiments, a hydrocracking feed stream including a hydrocarbon and a portion of the cracked stream including the fuel gas is contacted with a hydrocracking catalyst to form a hydrocracked stream including ethane.

The hydrocracked streams described herein can include unreacted components of the hydrocracking feed stream (e.g., the hydrocarbon and H₂), methane, ethane, propane, butane, and/or heavier hydrocarbons. In some embodiments, the hydrocracked stream includes up to about 3 wt. % of propane, and up to about 8 wt. % of butane. In some embodiments, at least a portion of the hydrocracked stream is further cracked in a steam cracking reactor (e.g., as described herein) to form ethylene. In some embodiments, the hydrocracked stream is diluted with additional ethane (e.g., from an external source) to form a steam cracking feed stream.

As described above, the hydrocracking feed streams and steam cracking feed streams include a hydrocarbon. In some embodiments, the hydrocarbon present in the steam cracking feed includes at least a portion of a hydrocracked stream described herein, e.g., including methane, ethane, propane, butane, heavier hydrocarbons, and/or unreacted hydrocarbons present in the hydrocracking feed stream.

In some embodiments, the hydrocarbon present in the hydrocracking feed stream includes a pyrolysis oil. In certain such embodiments, the pyrolysis oil includes at least a portion of the pyrolysis product of a plastic (e.g., a waste plastic). In certain such embodiments, the plastic includes a polyester such as polyethylene terephthalate (PET) and polycaprolactone, a polyolefin such as low-density polyethylene (LDPE), linear low density polyethylene (LLDPE) medium-density polyethylene (MDPE), high-density polyethylene (HDPE), and polypropylene (PP), a polyvinyl chloride (PVC), a polystyrene (PS), a polycarbonate, a polylactide, a polyether, a polyacrylate, an acrylonitrile rubber such as acrylonitrile butadiene styrene (ABS), styrene acrylonitrile resin (SAN), acrylonitrile styrene acrylate (ASA), and nitrile rubber (NBR), a fiberglass, a nylon, a polyurethane, any copolymer thereof, or any combination thereof. Suitable methods and systems for pyrolysis are known in the art. For example, in some embodiments, the pyrolysis includes thermal pyrolysis without a catalyst. In some embodiments, the pyrolysis includes catalytic pyrolysis. In some embodiments, the pyrolysis includes hydrothermal pyrolysis. In certain such embodiments, plastic (e.g., a waste plastic) and water are mixed in a reactor at high pressure (e.g., about 10 MPa) and mild temperature (e.g., about 300° C.) to form the pyrolysis oil.

In some embodiments, the pyrolysis oil includes a treated pyrolysis oil. In some embodiments, the treated pyrolysis oil includes at least a portion of the product of hydrotreatment of a raw pyrolysis oil (e.g., formed by pyrolysis of a waste plastic). For example, in certain such embodiments, a hydrotreating feed stream including a raw pyrolysis oil and hydrogen is contacted with a hydrotreating catalyst to form the treated pyrolysis oil. Suitable methods and systems for hydrotreating a pyrolysis oil are known in the art. In some embodiments, the hydrotreating feed stream includes at least a portion of the second electrolysis stream including hydrogen.

In some embodiments, a purification feed stream including a raw pyrolysis oil is washed to form the treated pyrolysis oil. Other suitable methods and systems for purifying a pyrolysis oil, e.g., by absorption or adsorption, are known in the art.

In some embodiments, the treated pyrolysis oil has a reduced olefin content as compared to the raw pyrolysis oil. In some embodiments, the treated pyrolysis oil has a reduced content of heteroatom- and/or metal-containing contaminants as compared to the raw pyrolysis oil. In some embodiments, the contaminant includes arsenic, calcium, chromium, iron, lead, aluminum, mercury, oxygen, zinc, potassium, silicon, sodium, copper, nickel, vanadium, sulfur, phosphorus, chlorine, fluorine, or any combination thereof. In some embodiments, the contaminants are present in the treated pyrolysis oil within industrial limits for cracking processes such as hydrocracking, steam cracking, and the like.

In some embodiments, a paraffin content of the pyrolysis oil (e.g., including the treated pyrolysis oil) is about 16 wt. % to about 42 wt. %, about 20 wt. % to about 40 wt. %, or about 20 wt. % to about 26 wt. %. In some embodiments, a naphthalene content of the pyrolysis oil (e.g., including the treated pyrolysis oil) is about 0 wt. % to about 22 wt. %, about 2 wt. % to about 22 wt. %, about 14 wt. % to about 22 wt. %, or about 2 wt. % to about 21 wt. %. In some embodiments, an olefin content of the pyrolysis oil (e.g., including the treated pyrolysis oil) is about 15 wt. % to about 50 wt. %, about 8 wt. % to about 48 wt. %, about 8 wt. % to about 42 wt. %, or about 16 wt. % to about 48 wt. %. In some embodiments, an aromatics content of the pyrolysis oil (e.g., including the treated pyrolysis oil) is about 0 wt. % to about 40 wt. %, about 3 wt. % to about 37 wt. %, or about 28 wt. % to about 39 wt. %. In some embodiments, a boiling point range of the pyrolysis oil (e.g., including the treated pyrolysis oil) is about 15° C. to about 600° C. In some embodiments, a hydrocarbon range of the pyrolysis oil (e.g., including the treated pyrolysis oil) is C₅ to C₅₅.

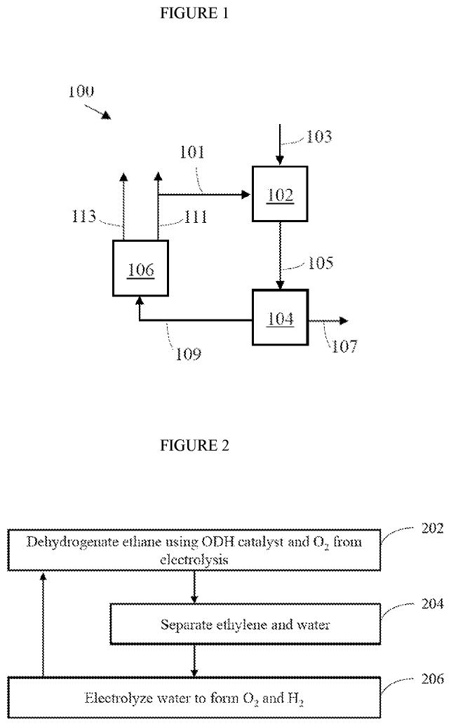

FIG. 2 is a process flow diagram of a method 200 for producing ethylene. The method starts at block 202 with the contacting of an oxidative dehydrogenation feed stream including ethane and oxidant stream including oxygen with an oxidative dehydrogenation catalyst to form a dehydrogenated stream including ethylene and water. At block 204, the dehydrogenated stream is separated to form a product stream including ethylene and a waste stream including water. At block 206, at least a portion of the waste stream including water is electrolyzed to form a first electrolysis stream including oxygen and a second electrolysis stream including hydrogen. The oxidant stream of block 202 includes at least a portion of the first electrolysis stream including oxygen.

### Definitions

The terms “a”, “an”, and “the” are used herein to include one or more than one unless the context clearly dictates otherwise. The term “or” is used to refer to a nonexclusive “or” unless otherwise indicated. The statement “at least one of A and B” has the same meaning as “A, B, or A and B”. In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, 3.3% to 4.4%) within the indicated range. The statement “about X to Y” has the same meaning as “about X to about Y”, unless indicated otherwise. Likewise, the statement “about X, Y, or about Z” has the same meaning as “about X, about Y, or about Z”, unless indicated otherwise.

As used herein, the term “about” can allow for a degree of variability in a value or range, for example, within 10%, within 5%, or within 1% of a stated value or of a stated limit of a range.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### EXAMPLES

The invention is further described in the following examples, which do not limit the scope of the invention described in the claims.

As shown in Table 1, below, integration of water electrolysis with oxidative dehydrogenation and, optionally, hydrocracking, can be economically feasible, as compared to processes where only hydrogen or oxygen are needed.

TABLE 1
Energy and CO₂ Emission of Water Splitting, O₂ Production (by
Cryogenic Air Separation), and H₂ Production (by Steam
Methane Reforming)

| | Energy | | Carbon | |
|---|---|---|---|---|
| | Demand | | Intensity (kg of | |
| | (kWh/kg of | | CO₂-eq/kg of | |
| | product) | | product) | |
| | H₂ | O₂ | H₂ | O₂ |
| Water | 48.9 | 6.2 | 0.79 | 0.10 |
| Splitter | | | | |
| SMR | 46.3 | — | 1.68 | — |
| ASU | — | 0.46 | — | 0.12 |

Four scenarios of the ethylene production process were modeled using Aspen Plus V11. Briefly, the Peng-Robinson method was utilized as a general equation of state for hydrocracking, air separation, steam-methane reforming, etc. The e-NRTL (ELECNRTL) method was utilized for water splitting, and IAPWS-95 and STEAM-TA were utilized for free water thermodynamic phase properties. Inlets were set to 25° C. and atmospheric pressure. The fuel gas included 73.253 wt. % H₂, 0.08 wt. % CO, 0.419 wt. % H₂O, 25.21 wt. % CH₄, 1.018 wt. % N₂, and 0.02 wt. % C₂H₄. Air included 79 mol % H₂ and 21 mol % O₂. Other parameters, including fuel cell efficiency, water splitter power consumption, steam methane reforming efficiency, and cryogenic air separation energy demand were selected based on convention.

### Example 1. Simulation of Integrated Ethylene Production Processes

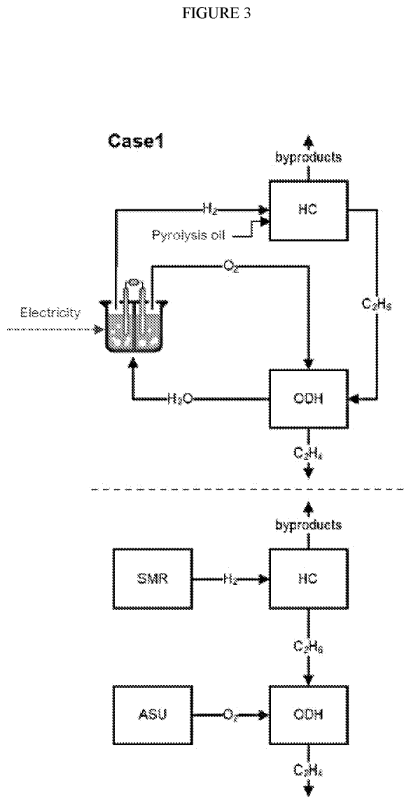

In Case 1, an ethylene production process including water electrolysis and oxidative dehydrogenation (ODH) integrated with pyoil hydrocracking (HC), as depicted in FIG. 3 (top), was compared to a corresponding comparative process including, in place of water electrolysis, steam-methane reforming and cryogenic air separation, as depicted in FIG. 3 (bottom).

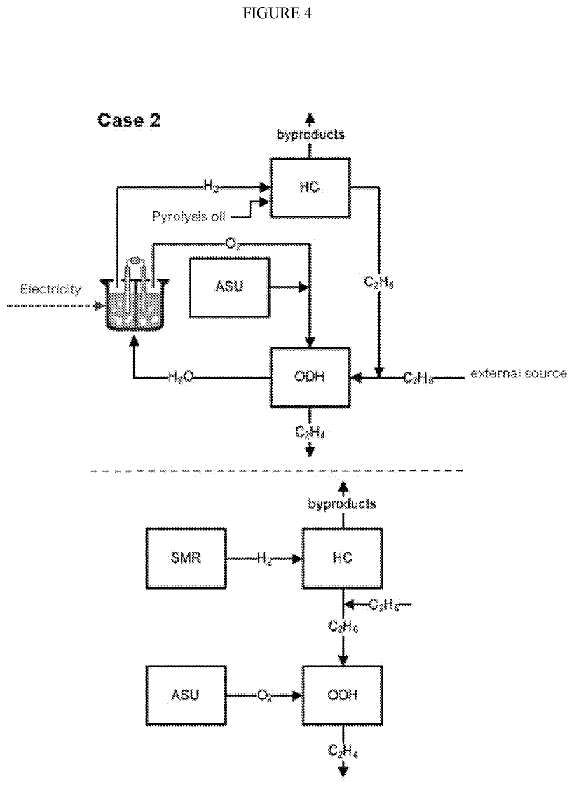

In Case 2, an ethylene production process including water electrolysis and oxidative dehydrogenation (ODH) integrated with cryogenic air separation (ASU) and pyoil hydrocracking (HC), as depicted in FIG. 4 (top), was compared to a corresponding comparative process including, in place of water electrolysis, steam-methane reforming and cryogenic air separation, as depicted in FIG. 4 (bottom). In Case 2, ethane supplemental to that produced by hydrocracking was available for oxidative dehydrogenation.

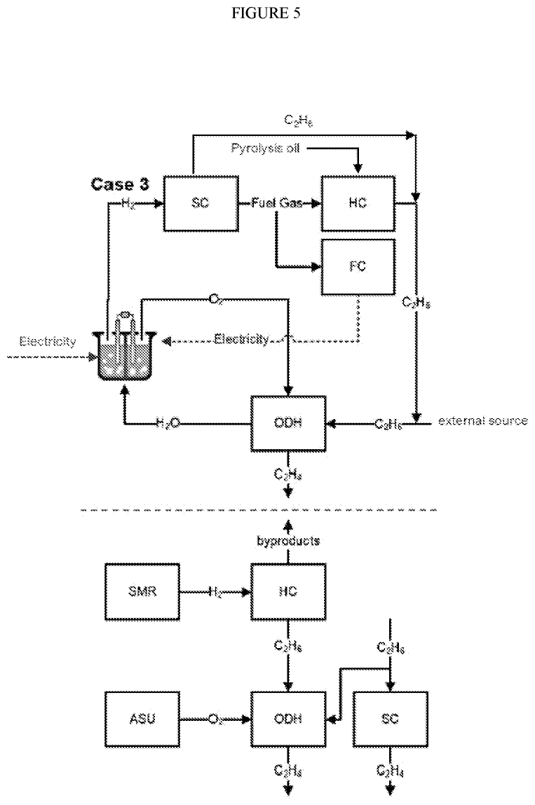

In Case 3, an ethylene production process including water electrolysis and oxidative dehydrogenation (ODH) integrated with pyoil steam cracking (SC), hydrocracking (HC), and a fuel cell (FC) providing electricity to the water electrolysis, as depicted in FIG. 5 (top), was compared to a corresponding comparative process including, in place of water electrolysis and the fuel cell, steam-methane reforming and cryogenic air separation, as depicted in FIG. 5 (bottom). In Case 3, ethane supplemental to that produced by hydrocracking was available for oxidative dehydrogenation.

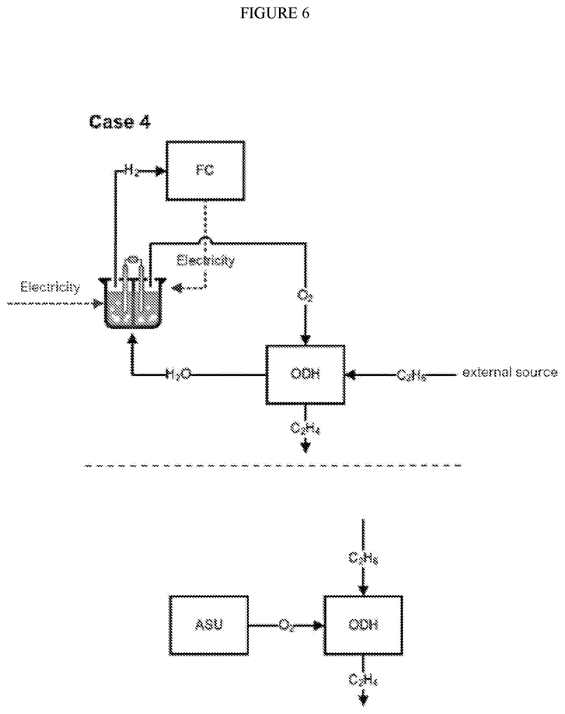

In Case 4, an ethylene production process including water electrolysis and oxidative dehydrogenation (ODH) integrated with a fuel cell (FC) providing electricity to the water electrolysis, as depicted in FIG. 6 (top), was compared to a corresponding comparative process including, in place of water electrolysis and the fuel cell, cryogenic air separation, as depicted in FIG. 6 (bottom). In Case 4, ethane supplemental to that produced by hydrocracking was available for oxidative dehydrogenation.

For each of Cases 1-4, separation trains were identical for the respective inventive and comparative simulations. To compare these cases, the energy requirements and carbon intensity were adjusted based on the production of one kilotonne of ethylene per year from oxidative dehydrogenation, depending on its oxygen consumption. The simulated oxidative dehydrogenation process required a feed of 224,066.6 kg/hr to yield 1,565.4 kta of ethylene. Because the product slate of hydrocracking supplements the ethane feed to the oxidative dehydrogenation process by 6,405.3 kg/hr, the need for fossil-based ethane feedstock is decreased. In energy and emission calculations, the ethylene yield was reported based on the oxygen requirement of oxidative dehydrogenation.

Tables 2-4, below, summarize the results of the simulation. Results were based on the following emissions assumptions: green hydrogen: 6.6 kg CO₂-eq/GJ H₂; oxygen: 0.115 kg CO₂-eq/kg of O₂; blue hydrogen (high performance plant): 14 kg CO₂-eq/GJ H₂; fuel gas: 19 kg CO₂-eq/GJ; electrical grid: 0.25 kg CO₂-eq/kWh. The results show that CO₂ emission was reduced in each of Cases 1-4, which included an integrated water splitter, as compared to the respective comparative processes. Utilizing water electrolysis as an alternative to SMR and cryogenic air separation also reduced the energy requirements in Cases 2 and 3, relative to the respective comparative processes. The computer simulations demonstrated that integration of hydrocracking and/or steam cracking processes, optionally in combination with a fuel cell, with water splitting can lower the energy requirements and environmental impacts of ethylene production, while also taking advantage of waste water from oxidative dehydrogenation.

TABLE 2
H₂ and O₂ Requirements and Ethylene Yield of
Simulated Cases 1-4

| | H₂ | O₂ | Ethylene |
|---|---|---|---|
| | Requirement | Requirement | Yield |
| Case | (kg/hr) | (kg/hr) | (kta) |
| 1 | 38,430.0 | 305,006.7 | 2,991.8 |
| 2 | 1,098.0 | 164,145.4 | 1,610.1 |
| 3 | 20,681.9 | 164,145.4 | 1,610.1 |
| 4 | 0.0 | 159,583.5 | 1,565.4 |

TABLE 3
Energy Requirements (MW) of Simulated Cases
1-4 per 1 kta of Ethylene Yield

| | Inventive | Comparative | |
|---|---|---|---|
| Case | Example | Example | Reduction |
| 1 | 0.6 | 0.6 | −1.44% |
| 2 | 0.1 | 0.1 | 1.03% |
| 3 | 0.4 | 0.6 | 29.25% |
| 4 | 0.4 | 0.0 | −681.80% |

TABLE 4
GHG Emission (kg of CO₂-eg/hr) of Simulated
Cases 1-4 per 1 kta of Ethylene Yield

| | Inventive | Comparative | |
|---|---|---|---|
| Case | Example | Example | Reduction |
| 1 | 10.2 | 27.9 | 63.53% |
| 2 | 11.6 | 12.9 | 9.54% |
| 3 | 13.4 | 65.8 | 76.69% |
| 4 | 10.5 | 11.7 | 10.78% |

Other implementations are also within the scope of the following claims.

1. A method of producing ethylene, the method comprising:
contacting an oxidative dehydrogenation feed stream comprising ethane and an oxidant stream comprising oxygen with an oxidative dehydrogenation catalyst to form a dehydrogenated stream comprising ethylene and water;
separating the dehydrogenated stream to form a product stream comprising ethylene and a waste stream comprising water; and
electrolyzing at least a portion of the waste stream comprising water to form a first electrolysis stream comprising oxygen and a second electrolysis stream comprising hydrogen;
wherein the oxidant stream comprises at least a portion of the first electrolysis stream comprising oxygen.

2. The method of claim 1, further comprising, before contacting the oxidant stream with the oxidative dehydrogenation catalyst, separating oxygen from air to form an air-separated stream comprising oxygen, wherein the oxidant stream comprises at least a portion of the air-separated stream comprising oxygen.

3. The method of claim 1, further comprising oxidizing at least a portion of a fuel cell feed stream comprising at least a portion of the second electrolysis stream comprising hydrogen to produce electrical energy, wherein electrolyzing at least a portion of the waste stream uses at least a portion of the electrical energy from oxidizing the fuel cell feed stream.

4. The method of claim 1, further comprising heating a steam cracking feed stream comprising a hydrocarbon and water to form a cracked stream comprising ethane and a fuel gas comprising hydrogen, wherein the oxidative dehydrogenation feed stream comprises a portion of the cracked stream comprising ethane.

5. The method of claim 4, further comprising combusting at least a portion of the second electrolysis stream comprising hydrogen to produce thermal energy, and using at least a portion of the thermal energy to heat the steam cracking feed stream.

6. The method of claim 4, further comprising oxidizing at least a portion of a fuel cell feed stream comprising a portion of the cracked stream comprising the fuel gas to produce electrical energy, wherein electrolyzing at least a portion of the waste stream uses at least a portion of the electrical energy from oxidizing the fuel cell feed stream.

7. The method of claim 4, wherein the fuel gas further comprises methane.

8. The method of claim 7, further comprising separating at least a portion of the methane from the fuel gas.

9. The method of claim 4, further comprising contacting a hydrocracking feed stream comprising a hydrocarbon and hydrogen with a hydrocracking catalyst to form a hydrocracked stream comprising ethane, wherein the steam cracking feed stream comprises at least a portion of the hydrocracked stream comprising ethane.

10. The method of claim 9, wherein the hydrocracking feed stream comprises at least a portion of the second electrolysis stream comprising hydrogen.

11. The method of claim 9 or claim 10, wherein the hydrocracking feed stream comprises at least a portion of the cracked stream comprising the fuel gas.

12. The method of claim 9, wherein the hydrocracking feed stream comprises a pyrolysis oil.

13. The method of claim 12, wherein the pyrolysis oil comprises a treated pyrolysis oil.

14. The method of claim 13, further comprising contacting a hydrotreating feed stream comprising a raw pyrolysis oil and a portion of the second electrolysis stream comprising hydrogen with a hydrotreating catalyst to form the treated pyrolysis oil.

15. The method of claim 14, further comprising pyrolyzing a plastic to form the raw pyrolysis oil.

16. The method of claim 15, wherein the pyrolyzing comprises thermal pyrolysis, hydrothermal pyrolysis, or catalytic pyrolysis.

17. The method of claim 3 or claim 6, wherein oxidizing the fuel cell feed stream comprises contacting the fuel cell feed stream with an anode catalyst of a fuel cell.

18. The method of claim 1, further comprising storing a portion of the first electrolysis stream comprising hydrogen, storing at least a portion of the second electrolysis stream comprising oxygen, or both.

19. The method of claim 1, further comprising:
combusting an oxy-fuel combustion mixture comprising a portion of the first electrolysis stream comprising oxygen, and a fuel feed stream comprising a combustible fuel to produce thermal energy and carbon dioxide;
converting at least a portion of the thermal energy to electrical energy; and
capturing at least a portion of the carbon dioxide.

20. The method of claim 1, further comprising contacting a gas stream comprising a pollutant with a portion of the first electrolysis stream comprising oxygen to oxidize at least a portion of the pollutant.

21. The method of claim 1, wherein electrolyzing at least a portion of the waste stream uses electrical energy from a renewable energy source or a reduced-emissions process.

22. A system for producing ethylene, the system comprising:
an oxidative dehydrogenation reactor configured to contact an oxidative dehydrogenation feed stream comprising ethane and an oxidant stream comprising oxygen with an oxidative dehydrogenation catalyst to form a dehydrogenated stream comprising ethylene and water;
a separator configured to separate the dehydrogenated stream to form a product stream comprising ethylene and a waste stream comprising water; and
an electrolyzer configured to electrolyze at least a portion of the waste stream comprising water to form a first electrolysis stream comprising oxygen and a second electrolysis stream comprising hydrogen, and to provide at least a portion of the first electrolysis stream to the oxidative dehydrogenation reactor.

23. The system of claim 22, further comprising an air separation unit configured to form an air-separated stream comprising oxygen, and to provide at least a portion of the air-separated stream to the oxidative dehydrogenation reactor.

24. The system of claim 22, further comprising a fuel cell configured to oxidize at least a portion of a fuel cell feed stream to produce electrical energy, and to provide at least a portion of the electrical energy to the electrolyzer.

25. The system of claim 22, further comprising a steam cracking reactor configured to heat a steam cracking feed stream comprising a hydrocarbon and water to form a cracked stream comprising ethane and a fuel gas comprising hydrogen, and to provide a portion of the cracked stream comprising the fuel gas to the oxidative dehydrogenation reactor.

26. The system of claim 25, further comprising a pressure swing adsorption unit configured to separate methane from the fuel gas.

27. The system of claim 25, further comprising a hydrocracking reactor configured to contact a hydrocarbon and hydrogen with a hydrocracking catalyst to form a hydrocracked stream comprising ethane, and to provide a portion of the hydrocracked stream comprising ethane to the steam cracking reactor.

28. The system of claim 27, further comprising a hydrotreating reactor configured to contact a hydrotreating feed stream comprising a raw pyrolysis oil and a portion of the second electrolysis stream comprising hydrogen with a hydrotreating catalyst to form a treated pyrolysis oil, and to provide a portion of the treated pyrolysis oil to the hydrocracking reactor.

29. The system of claim 28, further comprising a reactor configured to pyrolyze a plastic to form the raw pyrolysis oil.
